(19) United States
(12) Patent Application Publication
(10) Pub. No.: US 2019/0307793 A1
(43) Pub. Date: Oct. 10, 2019

(54) METHOD OF ENABLING MUSCLE RELAXATION

(71) Applicant: Sense Technology, Inc., Halifax, PA (US)

(72) Inventor: Joseph M. Evans, Export, PA (US)

(73) Assignee: Sense Technology, Inc., Halifax, PA (US)

(21) Appl. No.: 16/383,924

(22) Filed: Apr. 15, 2019

Related U.S. Application Data
(63) Continuation of application No. 15/752,343, filed on Feb. 13, 2018, now abandoned.
(60) Provisional application No. 62/204,622, filed on Aug. 13, 2015.

(51) Int. Cl. A61K 33/42; A61K 33/14; A61P 21/02; A61K 31/7076; A61K 33/06; A61K 33/00

(52) U.S. Cl. CPC A61K 33/42; A61K 33/14; A61P 21/02; A61K 2300/00; A61K 33/06; A61K 33/00; A61K 31/7076

(57) ABSTRACT

A method of application to human skin of a water solution or an emulsion of water and oil of metallic salts at concentrations from 1 to not more than 10 times the normal concentration found in the interstitial cellular space in humans, including the addition of ADP, ATP, and various manual, impulse, light or electrical therapy.

This application is based upon U.S. Provisional Application Ser. No. 62/204,622, filed Aug. 13, 2015, and hereby claims the priority thereof to which it is entitled.

### BACKGROUND OF THE INVENTION

The incidence of low back and other neuromusculoskeletal pain continues to increase, with low back pain being the leading cause of disability in the world(1)(2). In addition to the loss of quality of life for those who experience musculoskeletal pain the cost to both patients and society is significant and increasing:
The annual cost of chronic pain in the United States, including healthcare expenses (direct medical costs), lost income, and lost productivity, is estimated to be $635 billion. This is significantly higher than the estimated annual costs in 2010, dollars of heart disease ($309 billion), cancer ($243 billion), and diabetes ($188 billion).
Total estimated medical costs associated with back and neck pain, two of the commonest presentations of patients with chronic pain, increased by 65% between 1997 and 2005, to about $86 billion a year. Overall, pharmaceutical expenditures related to back and neck pain increased by 188% between 1997 and 2005, but costs associated with prescription narcotics rose by an astounding 423%.
Nationally, the estimated annual direct medical cost of low back pain is $30 billion. In addition, the impact of back pain is $100-200 billion in decreased wages and lost productivity.
Patients with chronic pain have more hospital admissions, longer hospital stays, and unnecessary trips to the emergency department.(3)

Most back pain encountered by clinicians in their practice is referred to as nonspecific or as being of “unknown origin.” Musculoskeletal pain resulting from trauma such as whiplash, repetitive strain injury and heavy lifting is relatively easy to understand; however, the cause and, therefore, best treatment for acute or chronic nonspecific musculoskeletal complaints is elusive.

The response to this problem by the allopathic profession has been to downplay the potential seriousness of the problem and to educate providers and patients.(4) Other healing arts (referred to hereafter as “manual therapies”) in the United States and elsewhere have developed terminology for the description of and procedures for treatment that are unique to each profession. Osteopaths refer to the problem as an “osteopathic lesion,” chiropractors as a “subluxation,’ physical therapists as “joint instability,” acupuncturists as “trigger points,” and massage therapists as “adhesions.” Each of these professions has also developed what they consider to be unique approaches to therapy for these conditions; “manipulation,” “adjustment,” “muscle strengthening,” “needling and acupressure,” and “deep massage.”

All techniques of manual therapy claim to reduce pain and even to provide a reversal of the underlying condition causing pain. However, despite numerous clinical trials comparing different approaches, there is no single methodology or technique that has achieved recognition as a primary or “first among equals” treatment. In fact there exist and are practiced hundreds of “named techniques” in the manual professions that have thousands of advocates who claim that their approach is superior to all others. This state of practice is testimonial to the fact that there is no universally recognized “best” treatment approach because there is no fundamental understanding of the cause of low back and other musculoskeletal pain.

All manual therapies have at least two features in common. The first is that individuals seeking treatment have defined themselves as patients. They have accepted that an authority in the treatment of their complaint can be of help. When they are examined by that authority, they are further reassured that they can be helped. This initial and vitally important step recruits the central nervous system (CNS) to assist in closing the gate that modulates the perception of pain, as predicted by the Gate Theory of Pain(5).

The second feature shared by all manual therapies is the application of proprioceptive afferent input to the CNS. The acupuncture needle, osteopathic manipulation, chiropractic adjustment, physical therapy mobilization and therapists massage all provide proprioceptive stimulation by mechanical pressure, impulse, impact, stretching and active and passive movement. These proprioceptive inputs presumably elicit responses from the Golgi tendon complex, muscle spindles and mechanoreceptors in joints, muscles, ligaments and fascia which participate in the restoration of proprioception to the CNS. This increase in proprioceptive input biases the summation of afferent inputs in the substantia gelatinosa toward domination by large fiber inputs, relieving the symptom of pain.

While all approaches to musculoskeletal therapy including simple encouragement provided to the patient by the allopathic profession are associated with improvement in the patients' perception of pain, the lack of a fundamental understanding of the cause of common low back and neck pain prevents the development of methods of treatment that are truly effective in reducing or eliminating the cause of pain. This is true whether the therapy consists of the adjustment, mobilization, massage, transcutaneous electrical nerve stimulation (TENS), micro-current, heat, “laser” therapy and, perhaps the classic case, trigger point therapy where initially an injection of anesthetic was used then needling only and finally pressure alone.

No prior art for the treatment of the underlying cause of non-specific musculoskeletal pain exists because the underlying cause of such pain is unknown.

### DESCRIPTION OF THE DRAWINGS

FIG. 1: is a diagram of the repetitive mechanism;

FIG. 2: is a diagram of the normal muscle contraction and relaxation cycle illustrated in FIG. 1; and

FIG. 3: is a diagram of the failure of the calcium pump which results in a disruption of the repetitive cycle illustrated in FIGS. 1 and 2.

### DETAILED DESCRIPTION OF THE INVENTION

In the absence of a fundamental understanding of the true cause of common low back and neck pain (usually referred to as “pain of unknown origin) there can be no invention of improved therapy or methodology for the improved treatment of such pain. Therefore, in order to create a truly useful invention, one that would be of benefit to therapists by providing improved patient results through the practice of the invention and benefit the patient through faster reduction in pain as well as reduction in relapses and improved general health, a new approach to the understanding of common musculoskeletal pain is required that identifies the true cause of such pain. The present invention is dependent on this approach and the invention cannot exist without this understanding.

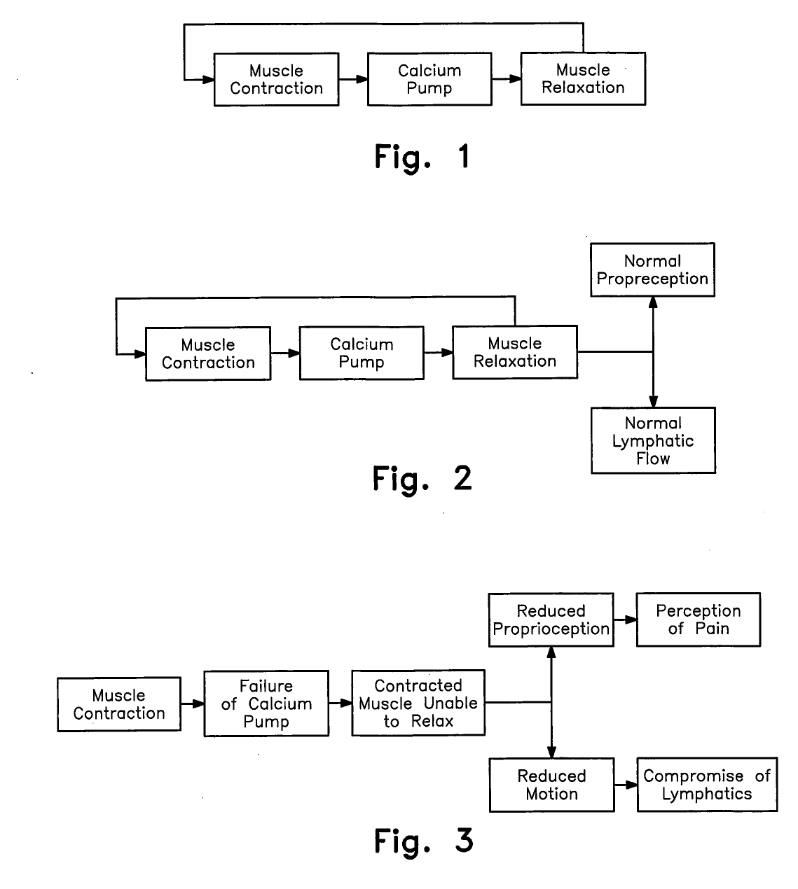

In order to develop an understanding of muscle dysfunction that results in pain, it is necessary to first understand normal pain-free muscle function. Fortunately, the fundamental physiology of normal muscle function is described in current physiology texts(6) and is generally recognized as an accurate portrayal of the contraction and, most importantly, the relaxation of muscle fibers. According to these texts, the cause of muscle fiber contraction is the release of calcium ions from the sarcoplasmic reticulum (a complex sac that interpenetrates the muscle) causes contraction of the myofibrils which results in contraction of the muscle fiber. Subsequent relaxation of the muscle fiber is enabled by the ATP energized calcium pump which returns calcium ions to the sarcoplasmic reticulum where they are available for the initiation of the next muscle contraction. This repetitive mechanism is illustrated in FIG. 1.

Normal Cycle of Muscle Contraction and Relaxation

As a result of and coincident with normal muscle contraction and relaxation, sensors within the muscle (Golgi tendon complex and muscle spindles) continuously send information to the CNS regarding muscle length, speed of contraction and force. These signals are collectively referred to as proprioception. In addition, the contraction and relaxation of the muscles of the body play a significant part in the maintenance of lymphatic drainage of the waste products of cellular metabolism from the body, see FIG. 2.

### Muscle Contraction Cycle Produces Lymphatic Flow as Well as Proprioception

If the calcium pump malfunctions, the muscle cannot relax. Interruption of the normal contraction and relaxation cycle of the muscle in turn interrupts the normal flow of proprioceptive input to the CNS as well as the normal flow of lymphatic fluid. Disruption of proprioceptive input to the CNS is perceived as pain and disruption of the lymphatic flow can result in metabolic failure at the cellular level as illustrated in FIG. 3.

### Diagram of Neurochemical Hypothesis

Therefore the cause of atraumatic muscle pain is:
An acute stage consisting of an initiating event wherein a portion of muscle mass in a normal contractile state is prevented from extending or relaxing due to a local failure of the calcium pump which results in the inability of the muscle to relax. The inability of the muscle to relax results in the perception of pain.
If the muscle dysfunction is not immediately corrected, the initial muscle dysfunction will result in chronic compromise of the lymphatic system resulting in potentially serious and poorly understood consequences for the health of the individual. Such consequences may include but are not limited to: localized edema, decreased blood flow, production of abnormal products of cellular metabolism and other potentially serious health effects.

Calcium ions are the primary mediator of contraction itself and therefore must be available in sufficient quantity for normal muscle contraction and relaxation. The release of calcium into the muscle fiber causes contraction of the myofibrils which results in contraction of the muscle fiber. Subsequent relaxation of the muscle fiber is enabled by the calcium pump which returns calcium ions to the sarcoplasmic reticulum where they are available for the initiation of the next muscle contraction. The function of the calcium pump is dependent upon the availability of adenosine triphosphate (ATP) which supplies the pumping energy. A failure of the calcium pump would account for the inability of a muscle fiber to relax. Such a failure could be due to a reduction in the synthesis of ATP for continued cycling of the ATP powered calcium pump.

ATP synthesis can be restricted by a pH that is either too acidic or basic. Other mechanisms may also account for reduced levels of ATP that would compromise the calcium pump. It is well known that minimum levels of potassium ion concentration are necessary to allow contracted muscle to relax (6). Other metallic ions such as sodium, potassium, magnesium, calcium and phosphorous play important roles not only as mediators of membrane depolarization and myofibril contraction, but as necessary modulators of the creation and utilization of ATP. Metallic ion concentrations may exhibit abnormal local concentrations which reduce the availability of ATP leading to the inability of small localized muscle volumes or entire muscles to relax.

Maintenance of normal physiologic levels of neuro-chemicals, especially the common metallic ions which are normally found as salts of chlorine as well as phosphate would provide protection against back pain by maintaining the proper environment for the regeneration of ATP and the normal function of the calcium pump. Therefore, in order to prevent the failure of the calcium pump, an adequate supply of Ca²⁺ ions as well as ATP must be maintained in the cellular interstitial space as well as the sarcoplasmic reticulum. The supply of ATP is known to be dependent on the synthesis of adenosine diphosphate (ADP) to ATP which cannot take place in an environment of too low or too high pH or in the face of deficiencies of the ions of calcium, potassium, sodium, phosphorus, magnesium, chlorine and other elements that are essential to the proper functioning of the calcium pump.

To ensure the availability of these elements when needed for the efficient removal of calcium from the muscle fiber after muscle contraction requires that any one element that is in a lower concentration than normal be supplemented with additional ions to remedy the insufficiency. The normal interstitial ion concentrations of these elements are 140 meq/L Na, 5 meq/L K, 5 meq/L Ca, 1 meq/L Mg, 2 meq/L HPO4.¹ This may be accomplished preferentially without knowledge of the specific deficiency by the topical application of a soluble solution of all of the ions important to proper function of the calcium pump. Topical application of such a solution will create a positive diffusion gradient for each of the required ions toward the site of the dysfunctional muscle which is the source of pain. This may be accomplished by the application of a water based solution of the required metallic ions directly or dispersed as an emulsion in a compatible oil based carrier to the body in the vicinity of the dysfunctional muscle. The concentration of any metallic ion which is lower than optimal will be preferentially increased as the diffusion gradient for that ion will be greater than ions present in normal concentrations. ¹http://classes.midlandstech.edu/carterp/Courses/bio211/chap26/chap26.htm

As a practical matter, any concentration of each of the above metallic ions above the normal concentration found in the body should be sufficient to enable improvement in the ability of the muscle to relax in the face of a deficit of that component. However, high concentrations of any of the metallic salts of these ions will result in noticeable deposition of solids on the surface of the skin where the lotion has been applied as the water component evaporates. These deposits can cause skin drying and irritation. To avoid this source of skin irritation, it has been found that the optimum levels of solution concentration may be achieved at a concentration of three times the concentration found in the interstitial spaces of the body and should not exceed ten times these concentrations.

As a practical matter, any concentration of each of the above metallic ions above the normal concentration found in the body should be sufficient to enable improvement in the ability of the muscle to relax in the face of a deficit of that component. However, high concentrations of any of the metallic salts of these ions will result in noticeable deposition of solids on the surface of the skin where the lotion has been applied as the water component evaporates. These deposits can cause skin drying and irritation. To avoid this source of skin irritation, it has been found that the optimum levels of solution concentration may be achieved at a concentration of three times the concentration found in the interstitial spaces of the body and should not exceed ten times these concentrations.

While the above procedure will result in pain relief due to muscle relaxation independent of other factors, the preferred embodiment of the invention is to apply the solution of metallic ions in concert with other forms of therapy, particularly physical or manual therapies. Therapies such as massage, manipulation, adjustment, ultrasound, transcutaneous electrical stimulation, light stimulation etc. are known to have the common result of stimulating the synthesis of ATP enhancing the efficiency and effectiveness of pain relief. Manual methods have the additional advantage of flushing of the lymphatic drainage of the involved musculature removing irritants generated by the effects of muscle dysfunction and normalization of metallic ion concentrations within the intracellular spaces of the muscular tissue. Even more effective is the combination of complementary therapies such as manipulation and electrical or light stimulation.

The current invention teaches the maintenance and enhancement of the calcium pump, a concept that has not until now been associated with muscle pain, especially the inability of muscle to relax due to the failure of the calcium pump. The purpose of the current invention is to maintain and enhance general muscle function and metabolic health.

1. A method of enabling muscle relaxation and restoring normal muscle function in humans after muscle fiber contraction caused by the release of calcium ions from the sarcoplastmic reticulum into the anterior of the muscle fiber causing concentration of myofibrils resulting in the contraction in the muscle fibers; a calcium pump existing in every muscle enables the removal of calcium ions from the interior of the muscle fiber; ensuring an adequate supply of ATP (Adenosine Triphosphate) and metallic salts which provide the energy for the calcium pump, the method solely consists of determining the concentration of metallic salts of 1 to not more than 10 times the normal concentration found in the interstitial cellular space in humans contained in skin overlying a muscle fiber; then topically applying to humans only a water solution of said metallic salts; thereby resulting in muscle relaxation and the restoring of normal muscle function.

2. The method of claim 1 to restore and maintain normal muscle function by restoring proprioceptive input to the central nervous system (CNS) to reduce the perception of pain.

3. The method of claim 1 to restore and maintain normal muscle function by restoring function of a lymphatic system.

4. The method of claim 1 wherein the water solution of metallic salts include ions of sodium (Na⁺), calcium (Ca⁺⁺), magnesium (Mg⁺⁺), potassium (K⁺), chloride (Cl⁻) and phosphorus (HPO⁻₄, H2PO⁻₃).

5. The method of claim 4 where phosphorus is provided in the form of phosphate ions as part of the ATP.

6. The method of claim 4 where phosphorus is provided in the form of phosphate ions as part of ADP (adenosine-diphosphate).
